(19) United States
(12) Plant Patent Application Publication
(10) Pub. No.: US 2003/0028936 P1
(43) Pub. Date: Feb. 6, 2003

(54) Raspberry plant named 'Claudia'

(76) Inventors: Harry J. Swartz, Laurel, MD (US); Joseph A. Fiola, Keedysville, MD (US); Herbert D. Stiles, Blackstone, VA (US); Brian R. Smith, River Falls, WI (US)

Correspondence Address: JAMES A. POULOS, III, UNIVERSITY OF MARYLAND, OFFICE OF TECHNOLOGY COMMERCIALIZATION, 6200 BALTIMORE AVENUE, SUITE 300, RIVERDALE, MD, 20737-1054, US

(21) Appl. No.: 09/917,241

(22) Filed: Jul. 30, 2001

(51) Int. Cl. A01H 5/00

(52) U.S. Cl. PLT/204

(57) ABSTRACT

The present invention is a new and distinct spring bearing red raspberry cultivar named ‘Claudia’ (also tested as KCE-1), which is capable of consistantly producing large numbers of fruit in the spring midseason, the fruit being larger than that of the standard cultivars. The cultivar is characterized by moderate suckering ability, small red thorns and stiff upright canes which are slow to grow in the spring and quick to stop growth in the fall. These traits, along with native cold hardiness traits, has resulted in excellent winter survivability in ‘Claudia’ fruit grown in the northern U.S., east of the Rocky Mountains. Because of the slow growth of primocanes and the upright habit of the flowering trusses, fruit is usually not shaded and well presented during harvest.

### FIELD OF THE INVENTION

This invention concerns a new and distinct cultivar of raspberry plant with a botanical name of Rubus ideaus L.

### DESCRIPTION OF RELATED PRIOR ART

Several cultivars of raspberry plant are known. For instance, raspberry cultivars named ‘Anne’, ‘Caroline’ and ‘Lauren’ have been described in U.S. Plant Pat. Nos. 10,411, 10,412 and 10,610, respectively. The new and distinct cultivar of the present invention is a raspberry plant named ‘Claudia’. This new and distinct cultivar of the present invention differs from ‘Anne’ in bearing red fruit in the spring, rather than golden fruit in the fall. ‘Claudia’ can be distinguished from ‘Caroline’ in that ‘Claudia’ is spring bearing, but ‘Caroline’ is fall bearing. ‘Claudia’ and ‘Lauren’ are both spring bearing red raspberry cultivars, but ‘Claudia’ can be distinguished from ‘Lauren’ in having a later ripeness period, much stouter canes and floricane trusses that are well exposed due to late primocane growth.

### ORIGIN OF THE NEW CULTIVAR

The new cultivar of spring bearing red raspberry originated from a controlled cross at the University of Maryland Greenhouses in College Park, Md.. The cross “CE” was ‘Skeena’ (unpatented) ×DCB-4 (unpatented) and was made in the winter of 1990. DCB-4 was a selection of the cross ‘AmosH’ (unpatented) ×NY817 (unpatented), made in 1983. This year was designated “K” as part of the University of Maryland at College Park; Rutgers University of New Brunswick, N.J.; Virginia Polytechnic Institute and State University, Southern Piedmont Agricultural Research and Education Center at Blackstone; and the University of Wisconsin at River Falls Cooperative Bramble Breeding program. The clone was first selected in 1993 at the Wye Research and Education Center of the University of Maryland located at Centerville, Md. and was therefore designated “-1”. Thus, the complete breeding designation was “KCE-1”.

### SUMMARY OF THE NEW CULTIVAR

This application relates to a new and distinct red fruited, spring bearing raspberry cultivar, botanically known as Rubus ideaus L. The following characteristics are outstanding:

1. The plant is very productive in the Northeastern United States due to its good cold hardiness and native productivity, including large fruit size.

2. Fruit is very well presented due to the stiffniess of flowering trusses and the lateness of the growth of primocanes, which do not reach the flowering canopy during harvest.

3. The stiffness and upright characteristic of its primocanes, which reduce the need for trellis support.

The following characteristics are useful in distinguishing this cultivar and can be useful for cultivar identification.

1. Plants are moderately suckering and very upright, growing to 4 feet or taller when mature.

2. Canes are very cold hardy, surviving temperatures of −20° F. in Wisconsin in two years.

3. Primocanes initiate growth very late in Spring, therefore fruit on floricanes are well presented (not shaded) during harvest.

4. Primocanes terminate growth early in the Fall, tip dieback is uncommon.

5. The fruit is produced in the midseason from the floricanes. Primocane produced fruit is rare.

### BRIEF DESCRIPTION OF THE PHOTOGRAPHS

The accompanying photographs show typical characteristics of the new variety:

FIG. 1 shows the floricane canopy of ‘Claudia’, in particular demonstrating the exposure of the fruit outside and above the primocane and the stiffness of flowering trusses.

FIG. 2 shows a ‘Claudia’ plant in fruit in June 1999, the tape is at 3 feet height. The plants are in their second growing season in Millersville, Pa.

FIG. 3 shows the type and density of thorns on an ‘Claudia’ primocane.

FIG. 4 shows a close-up coloring and size of ‘Claudia’ leaves and their long petiolule, the ruler is 6 inches long.

FIG. 5 shows a fruiting cluster of ‘Claudia’.

FIG. 6 shows development of ‘Claudia’ flowers and fruit.

FIG. 7 shows the size, uniformity, and color of harvested ‘Claudia’ fruit.

### DESCRIPTION OF THE NEW CULTIVAR

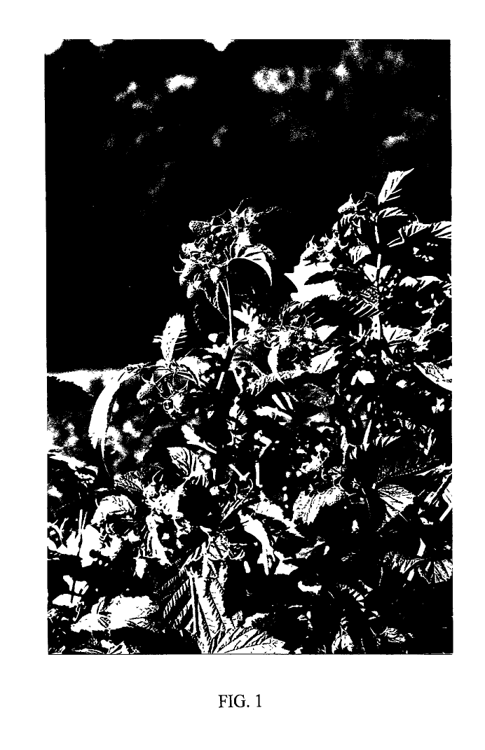

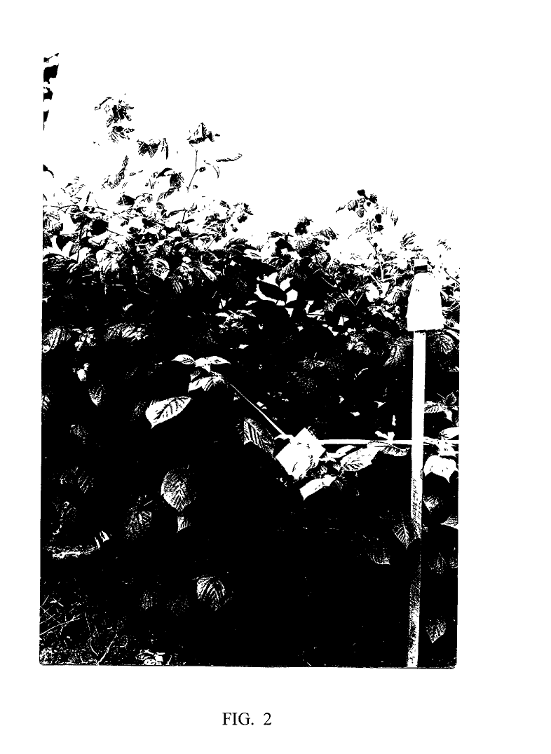

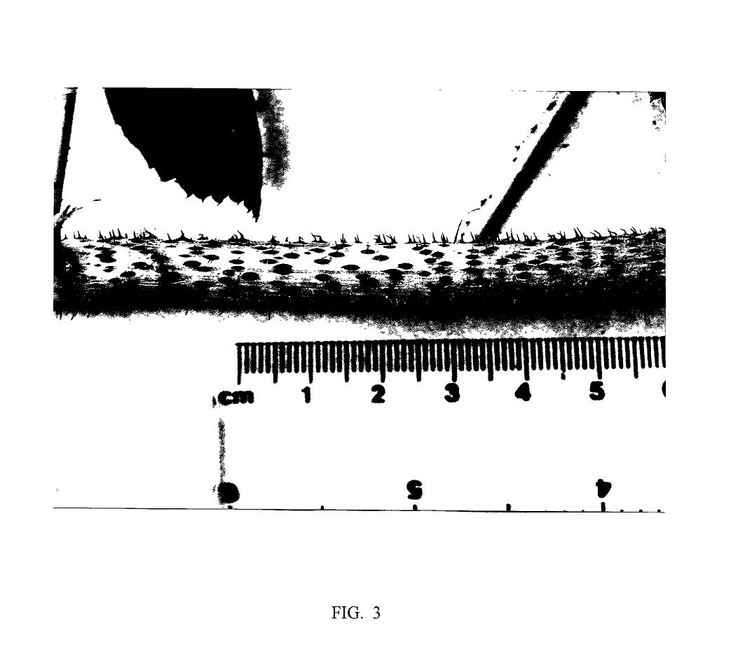

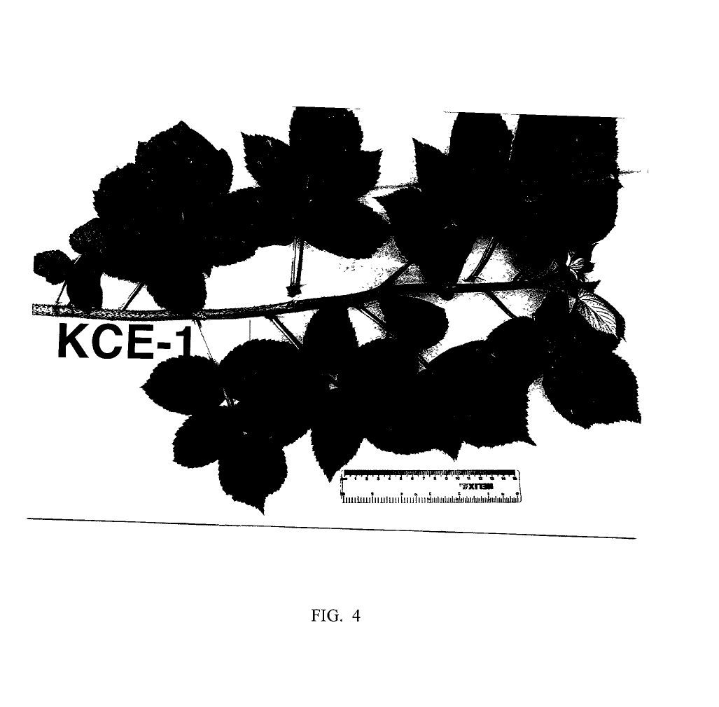

The following is a detailed description of the new cultivar, including fruit production, together with the cultivar's morphological characteristics. The characteristics of the cultivar were compared other standards used in the Mid-Atlantic Region of the U.S. The description is based on information provided by cooperating scientists from plants grown in fields at Cream Ridge and Colt's Neck, N.J., and Millersville, Pa., and from plants grown in the University of Maryland greenhouses at College Park, Md. ‘Claudia’ produces a moderate number of root- and crown-suckers, 53 per 10 ft of row on 3 year old plants grown in Colt's Neck, N.J. This number is similar to ‘Lauren’ (Plant Pat. No. 10,610), ‘Tulameen’ (unpatented) and “Sentry’ (unpatented), but greater than ‘Titan’ (Plant Pat. No. 5,404) and ‘AmosH’ (unpatented), typical cultivars tested or grown in the eastern United States. Unlike any other cultivar known to us, ‘Claudia’ primocanes are late in arising and elongating, thus penetrating the floricane canopy only after harvest (see FIG. 1 and 2). During the growing season, primocane and trusses from floricanes are light green colored (Royal Horticultural Society plate 143C) with a light red blush (Royal Horticultural Society plate 59A) in full sun, usually unbranched, very erect and moderately vigorous (see FIGS. 3, 4 and 5). Floricanes are moderately exfoliating at the base only and are brown in color, darkening from base to apex (Royal Horticultural Society plates 175A at the base and 175B at the apex). Thorns are moderate to high in density, 1 mm to 2 mm in length, stout and dark red (Royal Horticultural Society plate 59A) from their base to their apex (see FIGS. 3 and 4). The red coloration extends another 0.5 mm into the surrounding cane. Leaf upper surfaces are dark green, most closely in hue to Royal Horticultural Society Color Plate 137A, while the lower surface of the leaf is pubescent giving this surface a greyer color (Royal Horticultural Society plate 194B). Leaves are trifoliolate to pentafoliolate and average 12 cm from the distal end of the petiole to the distal end of the terminal leaflet (see FIG. 4). The cultivar has a slightly longer than average petiolule between the basal leaflets and more distal leaflet. The basal leaflets average 12 cm from terminal point to point. Leaf serration is common for most cultivars of red raspberry and cannot be used to distinguish this cultivar. Cane growth is very stiff and upright in the Mid-Atlantic States, unlike all other cultivars known to us, with the exception of the fall bearing cultivar ‘Heritage’. Canes will commonly terminate growth in the fall before the first killing frost. In this case, the apical most terminal bud will enlarge to give the appearance of a terminal bud. This is a rare trait for raspberries in the eastern U.S.

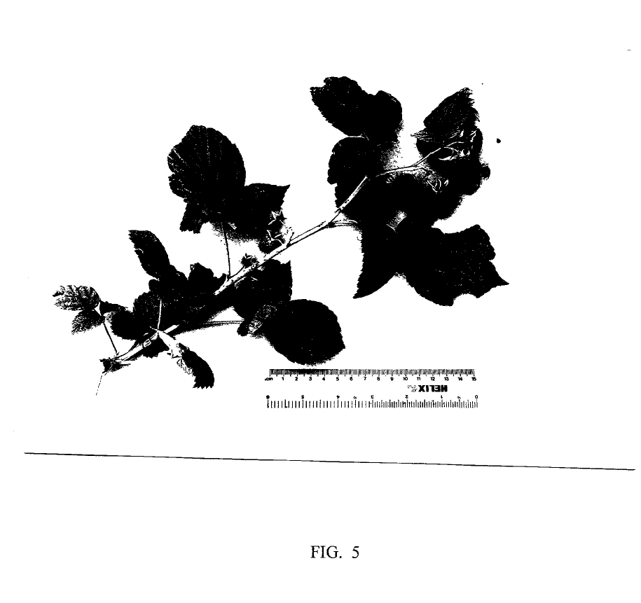

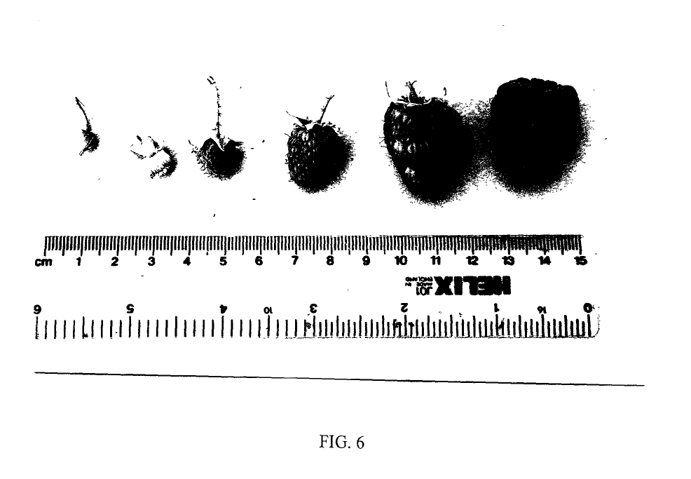

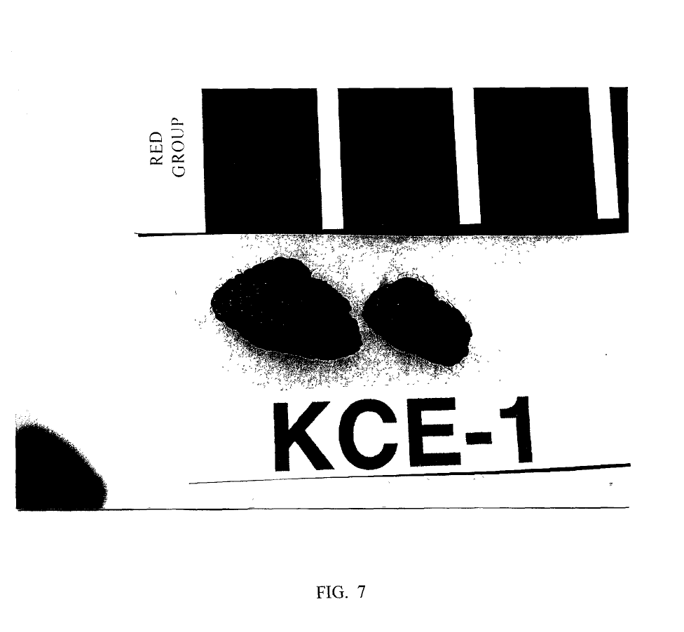

Fall fruit, that is, fruit borne on the top of the primocanes, is unknown in this cultivar. Floricanes rarely suffer cold injury in mid winter if temperatures fall below −20° F. or if a warm spring is interrupted by an unusual period of freezing temperatures. Canes can flower from all live buds in April to May depending on latitude, and fruit from mid June to late July in the eastern U.S. This ripeness period is 1-2 weeks later than ‘Lauren’ (U.S. Plant Pat. No. 10,610) and ‘Reveille’ (unpatented), but overlaps ‘Titan’ (U.S. Plant Pat. No. 5,404) and ‘Tulameen’ (unpatented). Flower morphology and early fruit morphology is typical of most red raspberry cultivars (unscented, 5 white petals 0.6 cm long resembling Royal Horticultural Society plate 155D which abscise within 4 days post pollination; 5 sepals resembling Royal Horticultural Society plate 194B and 1.1 cm long) and cannot be used to identify ‘Claudia’ (see FIGS. 5 and 6). Fruit trusses are very stiff and upright, and have typical cymose clusters with 6 to over 20 fruit well spaced out on a truss axis (see FIG. 5). After 25 days after pollination, fruit is easily distinguishable for this variety. Fruit has a very slight pubescence, producing a slightly glossy appearance. Fruit is decidedly conic, very large (see Table 1) and symmetrical. External and internal fruit color is red and typical of Royal Horticultural plate 46A (see FIG. 7). Drupelets are held together tightly. The collar is very uniform, although a collar knotch is common (see FIG. 7). During some environmental conditions, in some areas, fruit color varies slightly in different drupelets, that is across the fruit. The cavity width is similar to large-fruited cultivars and the drupelet thickness is higher than most cultivars (see FIG. 6). The fruit readily separates from the plant's recepticle, even when slightly unripe. The fruit does not break down after at least one week in common storage at 40° F. Flavor is mild to sweet and characteristic of red raspberry.

The plant is field resistant to many of the common pests and diseases in the eastern United States, e.g. mildew and verticillium wilt. The plant's reaction to Phytophthora fragarae root rot and late leaf rust is moderately susceptible, based on field reaction, not in controlled testing. Fruit is usually free from rot in the field.

### FRUIT PRODUCTION

‘Claudia’ has been tested in a grower trial in Millersville, Pa. The following data were collected in the summer from 1998 to 2000, the first year of production. Mid winter temperatures were below 0° F. in the winter of 1997-1998, resulting in complete cane death in ‘Tulameen no data (N.D.) was obtained for fruit size in 1998.

TABLE 1
Comparison of fresh fruit characteristics of ‘Claudia

| | Yield in lbs/acre | | | | Fruit Weight (grams) | |
|---|---|---|---|---|---|---|
| | 1998 | 1999 | 2000 | average | 1998 | 2000 |
| Claudia | 8650 | 5465 | 15696 | 9937 | 3.8 | 5.0 |
| Amos H | 7738 | 10475 | 9811 | 9341 | 3.0 | 3.8 |
| Tulameen | 0 | 7273 | 6268 | 4514 | N.D. | 4.6 |

‘Claudia has been asexually produced by tissue culture and field suckering since 1996 at South Deerfield, Mass. and at the University of Maryland, College Park, Md. Over that period, no as been observed or reported to us. Tissue culture explants were established tems and multiplication medium contained 3 to 15 micromolar benzyl adenine.

What is claimed is:

1. A new and distinct spring bearing red raspberry plant known as ‘Claudia’ as substantially described and illustrated herein.
